(19) United States
(12) Patent Application Publication
(10) Pub. No.: US 2020/0405531 A1
(43) Pub. Date: Dec. 31, 2020

(54) APPARATUS, SYSTEM, AND METHOD FOR AT LEAST ONE OF ICING, HEATING, AND COMPRESSING A USER'S BODY PART

(71) Applicant: Stephen Eugene Juaire, West Elbow Drive, FL (US)

(72) Inventor: Stephen Eugene Juaire, West Elbow Drive, FL (US)

(21) Appl. No.: 16/454,641

(22) Filed: Jun. 27, 2019

(51) Int. Cl. A61F 7/02; A41D 1/06

(52) U.S. Cl. CPC A61F 7/02; A61F 2007/0039; A41D 1/06

(57) ABSTRACT

A wearable device for at least one of icing, heating and compressing a user's body part is disclosed. The device includes an inner part and an outer part, each having an upper end and a lower end. The inner part has a first set of elastic properties, and the outer part has a second set of elastic properties. The outer part surrounds the inner part. A first attaching means is configured for attaching the lower end of the inner part to the lower end of the outer part. A compartment for receiving ice is defined by the space between the outer part and the inner part above the first attaching means. A compression force is provided by the second set of elastic properties compressing at least one of a hot element and cold element against the user's body part thereby eliminating a second attaching means above the first attaching means.

### CROSS-REFERENCE TO RELATED APPLICATIONS

Not applicable.

### STATEMENT REGARDING FEDERALLY SPONSORED RESEARCH OR DEVELOPMENT

Not applicable.

### INCORPORATION BY REFERENCE OF MATERIAL SUBMITTED ON A COMPACT DISC

Not applicable.

### TECHNICAL FIELD

The present invention relates to the field of therapeutic modalities, and more specifically, to wearable temperature and compression therapy devices for relieving pain and promoting healing of everyday aches and injuries.

### BACKGROUND

Cold therapy and heat therapy are useful adjuncts for the treatment of musculoskeletal injuries and soft tissue injuries. The goal of cold and heat therapy is to alter tissue temperature in a targeted region over time for the purpose of inducing a desired biological response. Static compression is often used in conjunction with cold and heat therapy for the care of acute injuries. To date, the primary reason for using compression is to increase external pressure on the tissue to prevent edema formation. This occurs by hindering fluid loss from the vessels in the injured area, making it more difficult for fluids to accumulate.

Cold therapy, also known as cryotherapy, is the application of any substance or physical medium to the body that removes heat, decreasing the temperature of the contact area and adjacent tissues. Cold therapy has multiple physiological effects on injured tissue. Decreasing temperatures of skin and muscle reduces blood flow to the cooled tissues by activating a sympathetic vasoconstrictive reflex. Cold-induced decreases in blood flow reduce edema and slow the delivery of inflammatory mediators, reducing inflammation of the affected area. Decreasing tissue temperature also reduces the metabolic demand of hypoxic tissues, potentially preventing secondary hypoxic damage in injured tissue. Many devices are available for application of cold therapy, including bags of crushed ice, and commercially available ice and gel packs.

Heat therapy, also known as thermotherapy, is the application of heat to the body resulting in increased tissue temperature. Heat therapy has multiple physiological effects on injured tissue. Rising temperatures of skin and muscle produce vasodilation that increases the supply of oxygen and nutrients and the elimination of carbon dioxide and metabolic waste. Additional physiological effects of heat therapy include increasing the extensibility of collagen tissues, decreasing joint stiffness, reducing pain, relieving muscle spasms, reducing inflammation, edema, and aids in the post-acute phase of healing. Superficial modes of heat therapy include hot water bottles, heat pads, and heated stones.

Traditional cold and heat therapy modalities can provide meaningful therapy but are often plagued by several challenges such as providing relief to only a specific area of the body. Individuals often need to simultaneously treat more than one soft tissue area within close proximity. For example, an injury to a hamstring area or group of muscles often includes the semimembranosus, semitendinosus, biceps femoris, quadratus femoris, and iliotibial band. As a result, the individual cannot treat the entirety of the injured area because the damaged soft tissue extends beyond the specific area covered by the therapeutic modality.

Moreover, individuals have a difficult time applying and maintaining therapeutic modalities in the desired position. For example, ice packs rarely stay in place due to their unconventional shape, and plastic bags of ice tend to leak and wet clothing. Additionally, compression is difficult to apply over therapeutic modalities. Therapeutic modalities require a protective layer of material to prevent the therapeutic modality from directly touching the skin and causing skin contact burns.

Therefore, a need exists to improve over the prior art and more particularly, for a therapeutic cold and heat delivery device that provides uniform pressure and temperature to an unrestricted combination of soft tissue groupings in the human body.

### SUMMARY

An apparatus, system, and method for at least one of icing, heating and compressing a user's body part is disclosed. This Summary is provided to introduce a selection of disclosed concepts in a simplified form that are further described below in the Detailed Description, including the drawings provided. This Summary is not intended to identify key features or essential features of the claimed subject matter. Nor is this Summary intended to be used to limit the claimed subject matter's scope.

In one embodiment, a wearable device for at least one of icing, heating and compressing a user's body part is disclosed. The wearable device includes an inner part having an inner part upper end and an inner part lower end defining an inner part length. The inner part has a first set of elastic properties and a first tubular shape such that the inner part is configured to fit snugly around a user's body part. The wearable device further includes an outer part having an outer part upper end and an outer part lower end defining an outer part length. The outer part has a second set of elastic properties and a second tubular shape. The outer part is arranged with the inner part such that the outer part surrounds the inner part. A first attaching means is configured for attaching the inner part lower end to the outer part lower end. A compartment is configured for receiving ice is defined by the space between the outer part and the inner part and above the first attaching means. A compression force is provided by the second set of elastic properties compressing at least one of a hot element and cold element against the user's body part thereby eliminating a second attaching means above the first attaching means. The second securing means being a structure other than the outer part for securing at least one of the hot element and cold element against the user's body part.

Additional aspects of the disclosed embodiment will be outlined in part in the description which follows, and in part will be obvious from the description, or may be learned by practice of the disclosed embodiments. The aspects of the disclosed embodiments will be realized and attained utilizing the elements, and combinations particularly pointed out in the appended claims. It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments. The embodiments illustrated herein are presently preferred, it being understood, however, that the invention is not limited to the precise arrangements and instrumentalities shown, wherein:

FIG. 1 is a front perspective side view of an apparatus for at least one of icing, heating and compressing a user's body part, according to an example embodiment of the present invention;

FIG. 2 is a side cross-sectional view of a system for at least one of icing, heating and compressing a user's body part, wherein a cold or hot element is located in a first area within the compartment; according to an example embodiment of the present invention;

FIG. 3 is a side cross-sectional view of a system for at least one of icing, heating and compressing a user's body part, wherein a cold or hot element is located in a second area within the compartment; according to an example embodiment of the present invention;

FIG. 4 is a side cross-sectional view of a system for at least one of icing, heating and compressing a user's body part, wherein a plurality of cold or hot elements are located in different areas within the compartment; according to an example embodiment of the present invention;

FIG. 5 is a side perspective view of an apparatus for at least one of icing, heating and compressing a user's body part, wherein a user is positioning a cold or hot element within the compartment; according to an example embodiment of the present invention;

FIG. 6 is a side cross-sectional view of a system for at least one of icing, heating and compressing a user's body part; according to an example embodiment of the present invention;

FIG. 7 is a side cross-sectional view of a system of an apparatus for at least one of icing, heating and compressing a user's body part, according to an example embodiment of the present invention; and

FIG. 8 is a flowchart describing the steps of the process for at least one of icing, heating, and compressing a user's body part, according to an example embodiment of the present invention.

### DETAILED DESCRIPTION

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions or modifications may be made to the elements illustrated in the drawings, and the methods described herein may be modified by substituting reordering or adding additional stages or components to the disclosed methods and devices. Accordingly, the following detailed description does not limit the disclosed embodiments. Instead, the proper scope of the disclosed embodiments is defined by the appended claims.

The present invention improves upon the prior art by providing uniform pressure and temperature to an unrestricted combination of soft tissue groupings in the human body. In other words, a therapeutic modality is not restricted to a particular location or region on a user's body. The wearable device includes a compartment for receiving ice defined by a space between an outer part and an inner part of the wearable device. A compression force is provided by a second set of elastic properties compressing at least one of a hot element and cold element against the user's body part thereby eliminating a second attaching means. As a result, the unrestricted application of a therapeutic modality provides a more effective treatment method because the wearable device conforms to the body and anchors the therapeutic modality precisely over the injury.

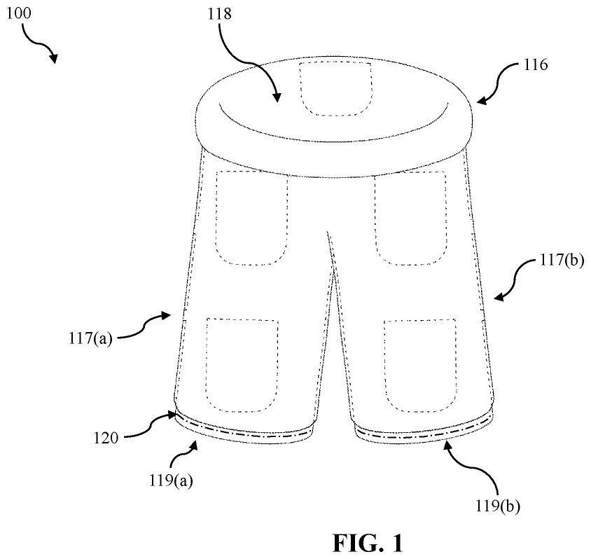

Referring now to the Figures, FIGS. 1-5 illustrate an apparatus for at least one of icing, heating and compressing a user's body part 101, and will be discussed together for ease of reference. The wearable device 100 comprises an inner part 105 and outer part 110. The outer part 110 is arranged with the inner part 105 such that the outer part surrounds the inner part. The inner part 105 includes an inner part upper end 106 and an inner part lower end 107, defining an inner part length. The inner part provides a protective insulation layer between a cold or heat modality and a user's skin. The inner part 105 has a first set of elastic properties and a first tubular shape such that the inner part 105 is configured to fit snugly around a user's body part. The outer part 110 includes an outer part upper end 111 and an outer part lower end 112 defining an outer part length. The outer part is comprised of a water-wicking material to draw moisture away from the body and prevent ice from leaking during treatment. The outer part 110 has a second set of elastic properties and a second tubular shape. The first set of elastic properties defines a first resilient force of between 17 mmHg-22 mmHg, and as described more fully below, when the wearable device 100 is in a fully sealed configuration, the second set of elastic properties defines a second resilient force of between 17 mmHg-22 mmHg. Elastic compression is the resilient force exerted by the wearable device on the user's body part. Compression is expressed in millimeters of mercury (mmHg). A higher number is correlated with a higher amount of compression while a lower number correlates with a lower compression.

In the present embodiment, the wearable device is a pair of shorts comprising a main body member 116 and a pair of leg sections 117(a), 117(b). The main body member is generally tubular for encircling the hip region of a user. The upper end of the main body member has an opening 118 to accommodate the hip region of a user. The main body member opening is defined by a flap 150 forming a seal at the upper end of the wearable device 100. The flap is defined by the inner part length being greater than the outer part length such that the inner part upper end 106 folds over the outer part upper part 111. When the wearable device 100 is in a fully sealed configuration, the flap 150 extends at least 1.0 inch from a top edge of the upper end of the outer part. The flap prevents water or perspiration from the heat element to escape the upper end of the main body. The lower end of the main body member has a pair of openings 119(a), 119(b) to accommodate the legs of the user and are appropriately sized and positioned so that the legs of the user may comfortably extend through the main body member.

The wearable device may be comprised of different flexible materials such as Lycra, polyester, cotton blend, nylon, similar blends, or any other suitable materials known in the art. More specifically, and without limitation, the material can be moisture absorbent, breathable, stretchable, meshed, or any blend or combination thereof. The material can also be thermoconductive to conduct heat better, or thermoreflective to help keep heat inside the wearable device. Further, the thickness and elasticity of fabric may be varied to accommodate different users and conditions. It should be appreciated that the wearable device can have other shapes and dimensions to accommodate men, women, and children of all sizes, and such variations are within the spirit and scope of the claimed invention.

The wearable device further includes a first attaching means 120 for attaching the inner part lower end 107 to the outer part lower end 112. The first attaching means is configured such that the therapeutic modality does not move below the lower ends. In the present embodiment, the first attaching 120 means comprises stitching the inner part lower end 107 to the outer part lower end 112. A plurality of different stitch patterns can be used including a chain stitch, straight stitch, and zigzag stitch, and such variations are within the spirit and scope of the claimed invention. Additionally, the stitching thread can be comprised of a natural fiber (cotton, wool, silk, linen) or a synthetic fiber (ravon, polyester, nylon). It should also be appreciated that other attachment means can be used to attach the inner part lower end 107 to the outer part lower end 112, including fasteners, snaps, zippers, ties, or any other suitable method known in the art.

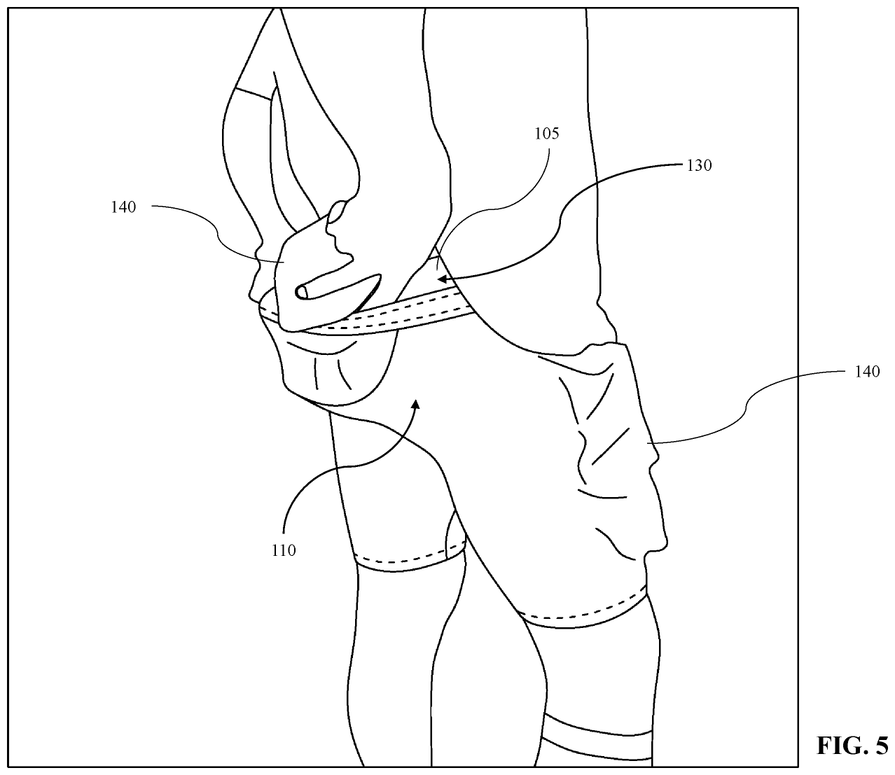

The wearable device further includes a compartment 130 defined by the space between the outer part 110 and the inner part 105, located above the first attaching means 120. It is understood that when the wearable device is worn by the user the inner part and the outer “sandwich” the cold element or hot element that has been inserted in the compartment. The inner layer may also provide an increased amount of friction to further prevent the cold or hot element from moving and facilitates maintaining the cold or hot element in a single position between the inner layer and outer layer unless acted on by a force greater than the inward compression force. The compartment is configured for receiving at least one of a hot element and cold element 140. The present invention improves upon the prior art by allowing the unrestricted application of the therapeutic modality to the user's body. In other words, the therapeutic modality is not limited or restricted to a particular location or region on the user's body. As best shown in FIG. 5, when the wearable device 100 is not in a fully sealed configuration, the therapeutic modality may be positioned and adjusted without removing, changing or adding layers within the compartment in an unlimited number of positions within the compartment spanning the entire wearable device. As a result, the unrestricted application of the therapeutic modality provides a more effective treatment method because the wearable device conforms to the body and anchors the therapeutic modality precisely over the injury.

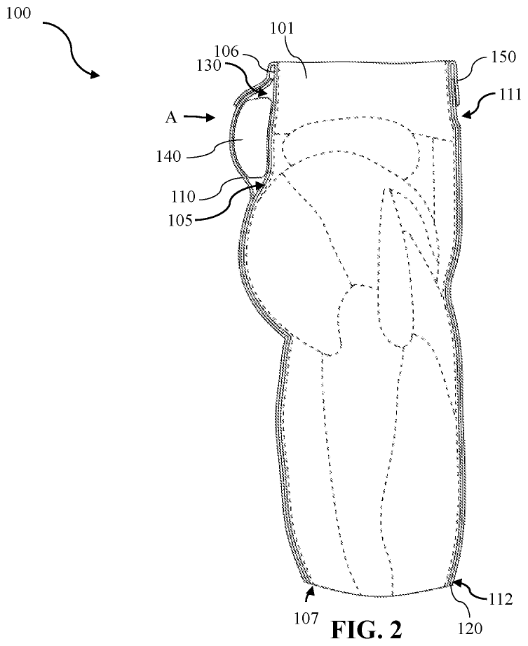

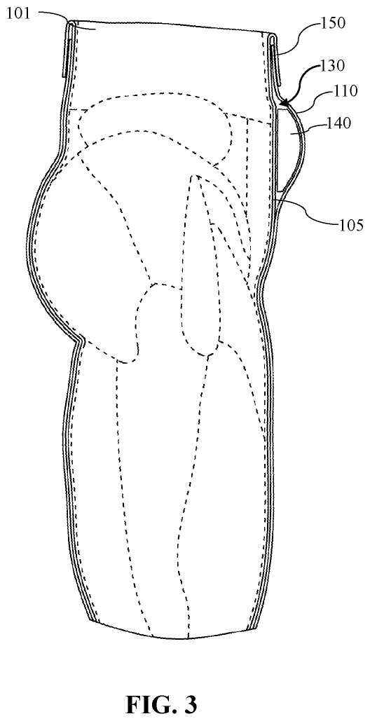

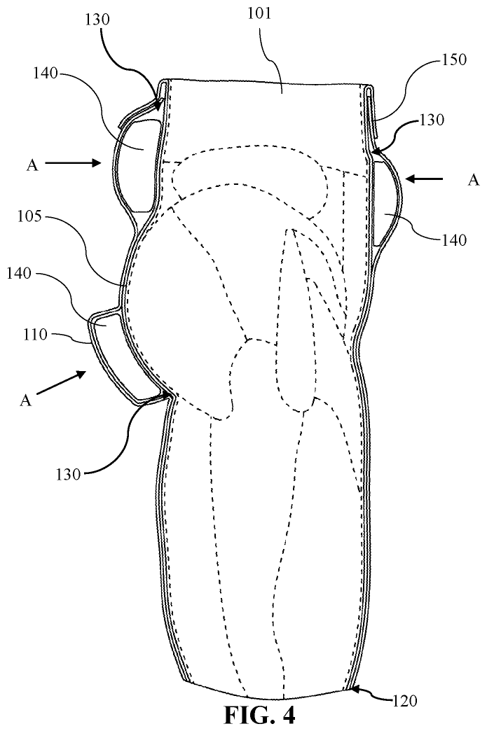

A compression force is provided by the second set of elastic properties compressing at least one of a hot element and cold element against the user's body part 101. The hot and cold elements may be comprised of crushed ice, commercially available ice and gel packs, hot water bottles, heat pads, and heated stones. However other devices or apparatus may be used for hot or cold elements that are within the spirit and scope of the present invention. The compression force provides an inward force (in the direction of line A) of at least 17 mmHg against the user's body part, thereby eliminating a second attaching means above the first attaching means 120. The second attaching means being a structure other than the outer part 110 for securing the at least one of the hot element and cold element against the user's body part 101, and may include a strap, button, pocket, hook and look fastener, snap, ridge or any combination thereof. In other words, the outer part having the second set of elastic properties eliminates the need for unnecessary pockets, straps of fasteners for securing the hot or cold element against the user's body. FIGS. 2-4 illustrates that the wearable device snugly fits the body of the user such that multiple therapeutic modalities located inside the compartment are anchored precisely over the desired area due to the snug fit of the wearable device. For example, in FIG. 2, one cold element 140 is positioned over the lower back (lumbar muscle) of the user 101, and in FIG. 3, one cold element positioned over the anterior wall (abdominal muscle) of the user 101. In FIG. 4, three cold elements are simultaneously positioned over the lower back (lumbar muscle), the anterior wall (abdominal muscle), and posterior pelvic region (gluteus maximus muscle) of the user 101. By eliminating a second attaching means above the first attaching means, the wearable device can be worn as any layer of clothing, including as an undergarment or as an over-garment, and permits the user to move, in moderation, without disturbing the precision of treatment. Additionally, by eliminating the second attaching means allows the user to position the hot element or cold element in a much greater amount of locations more easily that the existing prior art.

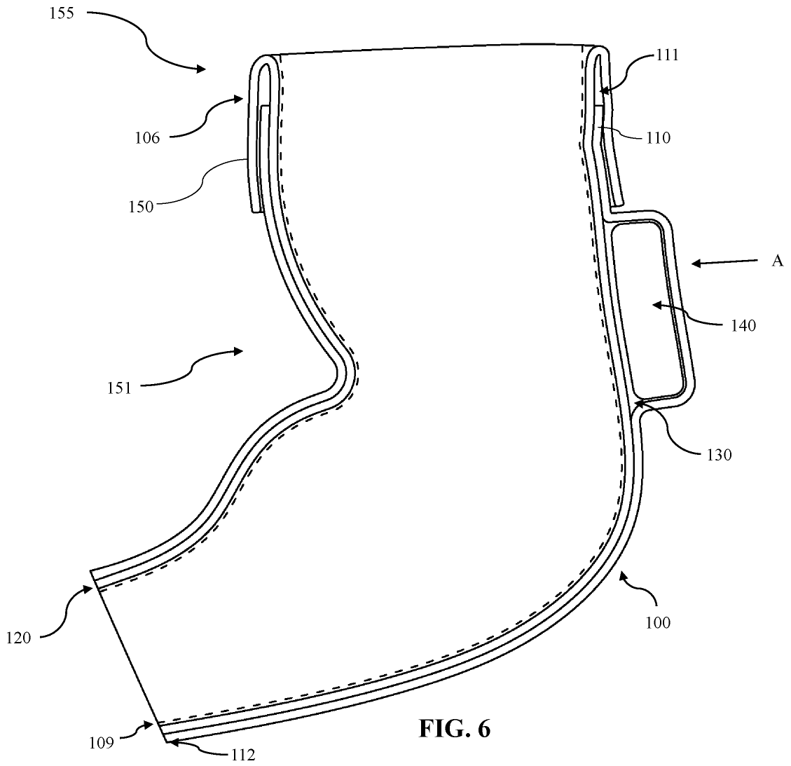

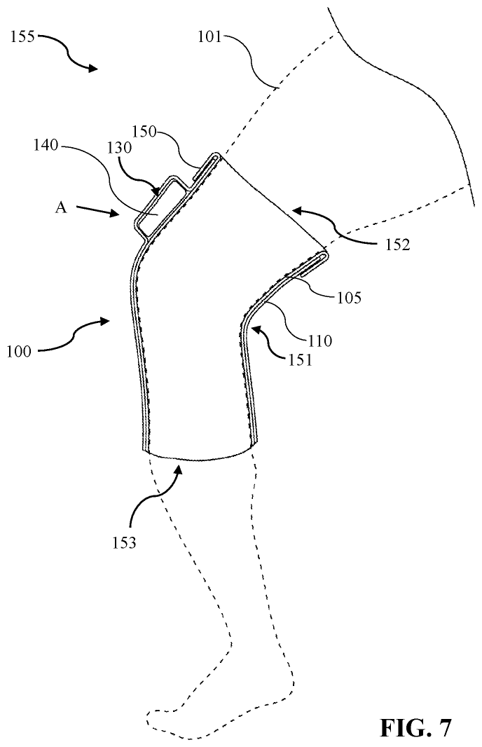

FIGS. 6-7 illustrate a system 155 for at least one of icing, heating, and compressing a user's body part 101 and will be discussed together for ease of reference. The system 155 includes a wearable device 100, and at least one of a hot element and a cold element positioned within a compartment on the wearable device. The wearable device 100 comprises an inner part 105 and outer part 110. The outer part 110 is arranged with the inner part 105 such that the outer part surrounds the inner part. The inner part 105 includes an inner part upper end 106 and an inner part lower end 107, defining an inner part length. The inner part provides a protective insulation layer between a cold or heat modality and a user's skin. The inner part 105 has a first set of elastic properties and a first tubular shape such that the inner part 105 is configured to fit snugly around a user's body part. The outer part 110 includes an outer part upper end 111 and an outer part lower end 112 defining an outer part length. The outer part may be comprised of a water-wicking material to draw moisture away from the body and prevent ice from leaking during treatment. However, it is understood that other types of materials may be used and are within the spirit and scope of the present invention. The outer part 110 has a second set of elastic properties and a second tubular shape. The first set of elastic properties defines a first resilient force of between 17 mmHg-22 mmHg, and as described more fully below, when the wearable device 100 is in a fully sealed configuration, the second set of elastic properties defines a second resilient force of between 17 mmHg-22 mmHg.

In another embodiment, the wearable device is a knee sleeve comprising a main body member 151. The main body member is generally tubular for encircling the knee region of the user 101. The upper end of the main body member has a first opening 152 to accommodate the upper knee region of the user. The first opening is defined by a flap 150 forming a seal at the upper end of the wearable device 100. The flap is defined by the inner part length being greater than the outer part length such that the inner part upper end 106 folds over the outer part upper part 111. When the wearable device 100 is in a fully sealed configuration, the flap 150 extends at least 1.0 inch from a top edge of the upper end of the outer part. The lower end of the main body member has a second opening 153 to accommodate the lower knee region of the user. The first and second openings are appropriately sized and positioned so that the leg of the user may comfortably extend through the first and second opening of the main body member.

The wearable device may be comprised of different flexible materials such as Lycra, polyester, cotton blend, nylon, similar blends, or any other suitable materials known in the art. More specifically, and without limitation, the material can be moisture absorbent, breathable, stretchable, meshed, or any blend or combination thereof. The material can also be thermoconductive to conduct heat better, or thermoreflective to help keep heat inside the wearable device. Further, the thickness and elasticity of fabric may be varied to accommodate different users and conditions. It should be appreciated that the wearable device can have other shapes and dimensions to accommodate men, women, and children of all sizes, and such variations are within the spirit and scope of the claimed invention.

The wearable device further includes a first attaching means 120 for attaching the inner part lower end 107 to the outer part lower end 112. The first attaching means is configured such that the therapeutic modality does not move below the lower ends. In the present embodiment, the first attaching 120 means comprises stitching the inner part lower end 107 to the outer part lower end 112. A plurality of different stitch patterns can be used including a chain stitch, straight stitch, and zigzag stitch, and such variations are within the spirit and scope of the claimed invention. Additionally, the stitching thread can be comprised of a natural fiber (cotton, wool, silk, linen) or a synthetic fiber (rayon, polyester, nylon). It should also be appreciated that other attachment means can be used to attach the inner part lower end 107 to the outer part lower end 112, including fasteners, snaps, zippers, ties, or any other suitable method known in the art.

The wearable device further includes a compartment 130 defined by the space between the outer part 110 and the inner part 105, located above the first attaching means 120. The compartment is configured for receiving at least one of a hot element and cold element 140. As best shown in FIG. 5, when the wearable device 100 is not in a fully sealed configuration, the therapeutic modality may be easily positioned directly over any injured area and adjusted without removing, changing or adding layers within the compartment in an unlimited number of positions within the compartment spanning the entire wearable device.

A compression force is provided by the second set of elastic properties compressing at least one of a hot element and cold element against the user's body part 101. The hot and cold elements may be comprised of crushed ice, commercially available ice and gel packs, hot water bottles, heat pads, and heated stones. The compression force provides an inward force (in the direction of line A) of at least 17 mmHg against the user's body part, thereby eliminating a second attaching means above the first attaching means 120. As mentioned, above, the second attaching means being a structure other than the outer part 110 for securing the at least one of the hot element and cold element against the user's body part 101, and may include a strap, button, pocket, hook and look fastener, snap, ridge or any combination thereof. FIG. 7 illustrates that the wearable device snugly fits the body of the user 101 such that a therapeutic modality located inside the compartment is anchored precisely over the knee area due to the snug fit of the wearable device. By eliminating a second attaching means above the first attaching means, the wearable device can be worn as any layer of clothing, including as an undergarment or as an over-garment, and permits the user to move, in moderation, without disturbing the precision of treatment. Additionally, the compression force provided by the second set of properties is configured such that the second attaching means is eliminated thereby allowing the user to position the hot element or cold element in a much greater amount of locations more easily that the existing prior art. Additionally as mentioned above the inner part may also provide an amount of friction thereby facilitating prevention of movement of the hot element or cold element when the element is positioned within the compartment and the wearable device is worn by the user.

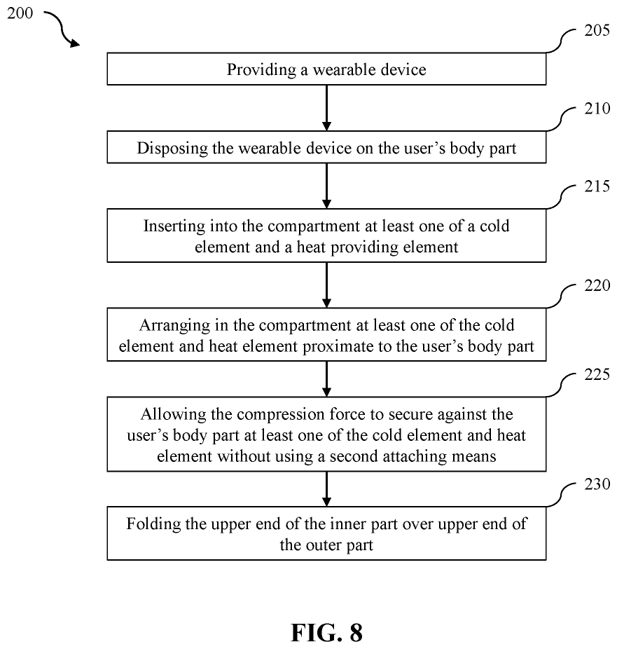

FIG. 8 is a flowchart describing the steps of the process for at least one of icing, heating, and compressing a user's body part, according to an example embodiment of the present invention. The sequence of steps depicted is for illustrative purposes only and is not meant to limit the method in any way as it is understood that the steps may proceed in a different logical order, additional or intervening steps may be included, or described steps may be divided into multiple steps, without detracting from the invention.

The process 200 begins with step 205, providing a wearable device. As described above, with respect to FIGS. 1-7, the wearable device 100 comprises an inner part 105 and outer part 110. The outer part 110 is arranged with the inner part 105 such that the outer part surrounds the inner part. The inner part 105 includes an inner part upper end 106 and an inner part lower end 107, defining an inner part length. The inner part provides a protective insulation layer between a cold or heat modality and a user's skin. The inner part 105 has a first set of elastic properties and a first tubular shape such that the inner part 105 is configured to fit snugly around a user's body part. The outer part 110 includes an outer part upper end 111 and an outer part lower end 112 defining an outer part length. The outer part may be comprised of a water-wicking material to draw moisture away from the body and prevent ice from leaking during treatment. The outer part 110 has a second set of elastic properties and a second tubular shape. The first set of elastic properties defines a first resilient force of between 17 mmHg-22 mmHg, and as described more fully below, when the wearable device 100 is in a fully sealed configuration, the second set of elastic properties defines a second resilient force of between 17 mmHg-22 mmHg. It is important that the second resilient force be sufficient such that the compression force or inward force provided by the second set of elastic properties is sufficient such that the hot element and cold element is maintained where it is positioned and such that the need for the second attaching means is eliminated. A flap is defined by the inner part length being greater than the outer part length such that the inner part upper end 106 folds over the outer part upper part 111. When the wearable device 100 is in a fully sealed configuration, the flap 150 extends at least 1.0 inch from a top edge of the upper end of the outer part. The wearable device further includes a first attaching means 120 for attaching the inner part lower end 107 to the outer part lower end 112. The first attaching means is configured such that the therapeutic modality does not move below the lower ends. A compartment 130 is defined by the space between the outer part 110 and the inner part 105, located above the first attaching means 120. The compartment is configured for receiving at least one of a hot element and cold element 140. A compression force is provided by the second set of elastic properties compressing at least one of a hot element and cold element against the user's body part 101. As shown in FIGS. 2-4, the compression force provides an inward force (in the direction of line A) of at least 17 mmHg against the user's body part, thereby eliminating a second attaching means above the first attaching means 120.

In step 210, the process requires disposing the wearable device on the user's body part. The user's body is positioned inward from a channel from the inner part, and the compartment is positioned outward from the user's body part. As shown in FIGS. 1-5, in one embodiment, the wearable device is a pair of shorts comprising a main body member 116 and a pair of leg sections 117(a), 117(b). The main body member is generally tubular for encircling the hip region of a user. The upper end of the main body member has an opening 118 to accommodate the hip region of a user. The lower end of the main body member has a pair of openings 119(a), 119(b) to accommodate the legs of the user and are appropriately sized and positioned so that the legs of the user may comfortably extend through the main body member.

As shown in FIGS. 6-7, in another embodiment, the wearable device is a knee sleeve comprising a main body member 151. The main body member is generally tubular for encircling the knee region of the user 101. The upper end of the main body member has a first opening 152 to accommodate the upper knee region of the user. The lower end of the main body member has a second opening 153 to accommodate the lower knee region of the user. The first and second openings are appropriately sized and positioned so that the leg of the user may comfortably extend through the first and second opening of the main body member.

In step 215, the process requires inserting into the compartment at least one of a cold element and a heat providing element. The hot and cold elements may be comprised of crushed ice, commercially available ice and gel packs, hot water bottles, heat pads, and heated stones. As best shown in FIG. 5, when the wearable device 100 is not in a fully sealed configuration, the at least one of the cold element and heat element may be positioned and adjusted without removing, changing or adding layers within the compartment in an unlimited number of positions within the compartment spanning the entire wearable device. Additionally, the present embodiment allows the user to position a hot element or cold element proximate to a user's body and within the wearable device in numerous positions without being limited by use of a fastener such as a hook, flap, snap, etc.

In step 220, the process requires arranging in the compartment at least one of the cold element and heat element proximate to the user's body part. For example, in FIG. 2, one cold element 140 is positioned over the lower back (lumbar muscle) of the user 101, and in FIG. 3, one cold element positioned over the anterior wall (abdominal muscle) of the user 101. In FIG. 4, three cold elements are simultaneously positioned over the lower back (lumbar muscle), the anterior wall (abdominal muscle), and posterior pelvic region (gluteus maximus muscle) of the user 101. Force (opposing the inward force in the direction of line A) may be applied to the outer layer to stretch the outer layer and enlarge the compartment so that the hot element or cold element may be positioned into the compartment. After positioning the hot element or cold element into the compartment, then the force stretching the outer layer to enlarge the compartment may be removed so that the compression force provided by the outer layer compresses the hot element or cold element against the user's body.

In step 225, the process requires allowing the compression force to secure against the user's body part at least one of the cold element and heat element without using a second attaching means. The second attaching means being a structure other than the outer part 110 for securing the at least one of the hot element and cold element against the user's body part 101, and may include a strap, button, pocket, hook and look fastener, snap, ridge or any combination thereof. As shown in FIGS. 2-4, the compression force provides an inward force (in the direction of line A) of at least 17 mmHg against the user's body part, thereby eliminating a second attaching means above the first attaching means 120. By eliminating a second attaching means above the first attaching means, the wearable device can be worn as any layer of clothing, including as an undergarment or as an over-garment, and permits the user to move, in moderation, without disturbing the precision of treatment. Additionally, by eliminating the second attaching means allows the user to position the hot element or cold element in a much greater amount of locations more easily that the existing prior art.

In step 230, the process requires folding the upper end of the inner part over the upper end of the outer part so that the flap is positioned outward from the outer part creating the seal. As discussed above, the flap is defined by the inner part length being greater than the outer part length such that the inner part upper end 106 folds over the outer part upper part 111. Thus, when the wearable device 100 is in a fully sealed configuration, the flap 150 extends at least 1.0 inch from a top edge of the upper end of the outer part.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

1. A wearable device for at least one of icing, heating and compressing a user's body part, the wearable device comprising:
an inner part having an inner part upper end and an inner part lower end defining an inner part length, wherein the inner part has a first set of elastic properties and a first tubular shape such that the inner part is configured to fit snugly around the user's body part;
an outer part having an outer part upper end and an outer part lower end defining an outer part length, wherein the outer part has a second set of elastic properties and a second tubular shape, wherein the outer part is arranged with the inner part such that the outer part surrounds the inner part;
a first attaching means for attaching the inner part lower end to the outer part lower end;
a compartment for receiving a therapeutic modality, the therapeutic modality comprising at least one of a cold element or a hot element;
wherein the compartment is defined by a space between the outer part and the inner part and above the first attaching means;
wherein the compartment spans the entire wearable device;
wherein a location of the compartment is fixed relative to the wearable device;
wherein, due to the compartment spanning the entire wearable device, the therapeutic modality is not restricted to a particular location on the user's body part; and,
a compression force provided by the second set of elastic properties compressing the therapeutic modality against the user's body part thereby eliminating a second attaching means above the first attaching means, wherein the second attaching means being structure other than the outer part for securing the therapeutic modality against the user's body part;
wherein a flap forming a seal at a wearable device upper end defined by the inner part length being greater than the outer part length such that an inner part upper end folds over the outer part upper part; and
wherein when the wearable device is in a fully sealed configuration, the flap extends at least 1.0 inch from a top edge of the outer part upper end.

2.-3. (canceled)

4. The wearable device of claim 1, wherein the first set of elastic properties defines a first resilient force of between 17 mmHg-22 mmHg.

5. The wearable device of claim 1, wherein the second set of elastic properties defines a second resilient force of between 17 mmHg-22 mmHg.

6. The wearable device of claim 1, wherein the compression force is providing an inward force of at least 17 mmHg against the user's body part.

7. The wearable device of claim 1, wherein the first attaching means comprises stitching attaching the inner part lower end to the outer part lower end.

8. The wearable device of claim 1, wherein the second attaching means comprises a strap, button, pocket, hook and look fastener, snap, ridge or any combination thereof.

9. A system for at least one of icing, heating and compressing a user's body part, the system comprising:
a wearable device having;
an inner part having an inner part upper end and an inner part lower end defining an inner part length, wherein the inner part has a first set of elastic properties and a first tubular shape such that the inner part is configured to fit snugly around the user's body part;
an outer part having an outer part upper end and an outer part lower end defining an outer part length, wherein the outer part has a second set of elastic properties and a second tubular shape, wherein the outer part is arranged with the inner part such that the outer part surrounds the inner part;
a first attaching means for attaching the inner part lower end to the outer part lower end;
a compartment for receiving a therapeutic modality, the therapeutic modality comprising at least one of a hot element and a cold element;
wherein the compartment is defined by a space between the outer part and the inner part and above the first attaching means;
wherein the compartment spans the entire wearable device;
wherein, due to the compartment spanning the entire wearable device, the therapeutic modality is not restricted to a particular location on the user's body part;
a compression force provided by the second set of elastic properties compressing the therapeutic modality against the user's body part thereby eliminating a second attaching means above the first attaching means;
wherein the second attaching means being structure other than the outer part for securing the therapeutic modality against the user's body part;
a flap forming a seal at a wearable device upper end defined by inner part length being greater than the outer part length such that an inner part upper end folds over the outer part upper part; and,
the therapeutic modality positioned within the compartment.

10. The system of claim 9, wherein when the wearable device is in a fully sealed configuration, the flap extends at least one inch from a top edge of the outer part upper end.

11. The system of claim 9, wherein the first set of elastic properties defines a first resilient force between 17 mmHg-22 mmHg.

12. The system of claim 9, wherein the second set of elastic properties defines a second resilient force between 17 mmHg-22 mmHg.

13. The system of claim 9, wherein the compression force is providing an inward force at least 17 mmHg against the user's body part.

14. The system of claim 9, wherein the first attaching means comprises stitching attaching the inner part lower end to the outer part lower end.

15. The system of claim 9, wherein the second attaching means comprises a strap, button, pocket, hook and look fastener, snap, ridge or any combination thereof.

16. A method for at least one of icing, heating and compressing a user's body part, the method comprising:
a) providing a wearable device, wherein the wearable device comprises,
an inner part having an inner part upper end and an inner part lower end defining an inner part length, wherein the inner part has a first set of elastic properties and a first tubular shape such that the inner part is configured to fit snugly around the user's body part;
an outer part having an outer part upper end and an outer part lower end defining an outer part length, wherein the outer part has a second set of elastic properties and a second tubular shape, wherein the outer part is arranged with the inner part such that the outer part surrounds the inner part;
a first attaching means for attaching the inner part lower end to the outer part lower end;
a compartment for receiving a therapeutic modality defined by a space between the outer part and the inner part and above the first attaching means;
wherein the therapeutic modality is at least one of a hot element and a cold element;
wherein the compartment spans the entire wearable device;
wherein, due to the compartment spanning the entire wearable device, the therapeutic modality is not restricted to a particular location on the user's body part; and,
a compression force provided by the second set of elastic properties compressing the therapeutic modality against the user's body part thereby eliminating a second attaching means above the first attaching means, wherein the second attaching means being structure other than the outer part for securing the therapeutic modality against the user's body part;
b) disposing of the wearable device on the user's body part;
wherein the user's body is positioned inward from a channel from the inner part;
wherein the compartment is positioned outward from the user's body part;
c) inserting, into the compartment, the therapeutic modality;
d) arranging in the compartment the therapeutic modality proximate to the user's body part;
e) allowing the compression force to secure against the user's body part the therapeutic modality without using a second attaching means; and,
f) folding the inner part upper end over the outer part upper end so that a flap is positioned outward from the outer part creating a seal.

17. (canceled)

18. The method of claim 16, wherein the second set of elastic properties defines a second resilient force between 17 mmHg-22 mmHg.

19. The method of claim 16, wherein the compression force is providing an inward force at least between 17 mmHg against the user's body part.

20. The method of claim 16, wherein the second attaching means comprises a strap, button, pocket, hook and look fastener, snap, ridge or any combination thereof.
